(19) United States
(12) Patent Application Publication
(10) Pub. No.: US 2007/0244467 A1
(43) Pub. Date: Oct. 18, 2007

(54) Self-Filling Two Chamber Injectable Device

(75) Inventors: Solomon S. Steiner, Mount Kisco, NY (US); Erik Steiner, New York, NY (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Biodel, Inc., State of Incorporation Delaware

(21) Appl. No.: 11/536,382

(22) Filed: Sep. 28, 2006

Related U.S. Application Data
(60) Provisional application No. 60/721,475, filed on Sep. 28, 2005.

(51) Int. Cl. A61M 37/00; A61M 31/00

(52) U.S. Cl. 604/518; 604/87

(57) ABSTRACT

A device has been designed which contains a chamber for diluent; a chamber for lyophilized or powdered medicament; and means for separating the medicament from the diluent. Suitable means for separation include a frangible disk and a separate container comprising the diluent. In the preferred embodiment, the medicament is lyophilized in the chamber and sealed under vacuum. In another embodiment, a movable piston acts as the seal. In still another embodiment, the diluent is packaged in a separate container under pressure. The two containers are connected by a tube, such as a nozzle or a needle. The tube can contain a dispersion or turbulence device (herein referred to as a “dispersion tube”). The dispersion tube has a one-way valve permitting flow of liquid from the pressurized container to the second container. When the user applies a force to the device such as a twisting or a downward push, the two containers are connected through the tube, and the contents of the pressurized container flow turbulently into the second container, ensuring thorough mixing. In the second embodiment, the movable piston is pushed to its full excursion by the incoming fluid. The diluent container and the connecting tube are then separated from the now filled cartridge, containing the dissolved medicament, under sterile conditions. The cartridge is placed in a pen device and is ready for use. The device described herein can be used for shipment, storage and use of unstable medicaments at room temperature. Suitable medicaments include, but not limited to peptide and proteins, DNA, RNA, antibodies and enzymes; preferably the medicament is insulin.

### CROSS-REFERENCE TO RELATED APPLICATION

This application claims priority to U.S. Ser. No. 60/721,475 entitled “Self filling Two Chamber Injectable Device” filed Sep. 28, 2005, by Solomon S. Steiner and Erik Steiner.

### FIELD OF THE INVENTION

The present invention is generally in the field of devices for dissolving and/or suspending a lyophilized medicament.

### BACKGROUND OF THE INVENTION

One of the many advantages of administering a medicament by injection is control of the dose administered. A good example of this need is the tight control of the dose of insulin required to adequately treat diabetes, where a difference of 0.03 ml in the dose can result in the difference between good glycemic control and hypoglycemia. To improve the accuracy and convenience of self-injection, numerous devices, frequently described as “insulin pens”, are currently available that allow the patient to dial in the dose with a high degree of accuracy. The medicaments for the devices are typically provided as liquid pre-filled disposable cartridges.

Since many injectables are unstable in aqueous solution or suspension, they either cannot be administered at all with the current devices, as is the case with glucagons, or their shelf life is markedly reduced, as is the case with insulin. Furthermore, the in aqueous solutions or suspensions containing the medicament frequently require refrigeration for both shipment and storage.

It is therefore an object of the present invention to provide devices which can be used for shipment, storage and use of unstable medicaments at room temperature.

### SUMMARY OF THE INVENTION

A device has been designed which contains a chamber for diluent; a chamber for lyophilized or powdered medicament; and means for separating the medicament from the diluent. Suitable means for separation include a frangible disk and a separate container comprising the diluent. In the preferred embodiment, the medicament is lyophilized in the chamber and sealed under vacuum. In another embodiment, a movable piston acts as the seal. In still another embodiment, the diluent is packaged in a separate container under pressure. The two containers are connected by a tube, such as a nozzle or a needle. The tube can contain a dispersion or turbulence device (herein referred to as a “dispersion tube”). The dispersion tube has a one-way valve permitting flow of liquid from the pressurized container to the second container. When the user applies a force to the device such as a twisting or a downward push, the two containers are connected through the tube, and the contents of the pressurized container flow turbulently into the second container, ensuring thorough mixing. In the second embodiment, the movable piston is pushed to its full excursion by the incoming fluid. The diluent container and the connecting tube are then separated from the now filled cartridge, containing the dissolved medicament, under sterile conditions. The cartridge is placed in a pen device and is ready for use. The device described herein can be used for shipment, storage and use of unstable medicaments at room temperature. Suitable medicaments include, but not limited to peptide and proteins, DNA, RNA, antibodies and enzymes; preferably the medicament is insulin.

### BRIEF DESCRIPTION OF THE DRAWINGS

FIG. 1 is a perspective view of a device including a compartment for lyophilized or powdered drug and diluent, separated by a frangible disk.

FIGS. 2A and 2B are perspective views of a device having a connecting tube which can be used to supply diluent directly into the lyophilized medicament chamber.

### DETAILED DESCRIPTION OF THE INVENTION

Devices which can be used for shipment, storage and use of medicaments are described herein. These devices are particularly useful for medicaments that are unstable at room temperature. Suitable medicaments include, but not limited to peptide and proteins, DNA, RNA, antibodies and enzymes; preferably the medicament is insulin. Preferably the device is designed to mix a single dose of diluent and lyophilized or powdered medicament.

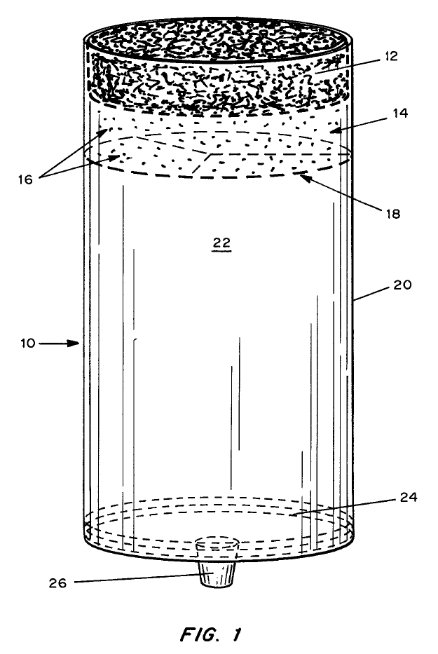

As shown in FIG. 1, device 10 consists of a closed, generally tubular system, containing a cartridge 20, which contains a movable piston 12, friable chamber 14 containing lyophilized or powdered medicament 16, a diluent chamber 22, and frangible disk 18 separating the lyophilized medicament 16 from the diluent 23 in the cartridge 20. In the preferred embodiment, the medicament is lyophilized in the friable chamber and sealed under vacuum. Alternatively, the movable piston may act as the seal. The device 10 can be attached to another device (not shown) by screw threads 24 for connection via the nozzle 26. Typically, the diluent 23 is added to the device 10 from another container, which can be a syringe or pre-pressurized container. Upon connection of the container containing the diluent 23 with the device 10, the diluent 23 transfers from the container into the device 10 (not shown in figures).

The cartridge can placed in a suitable device for administering the medicament to a patient, such as a pen device. To mix the diluent 23 with the medicament 16 and force the medicament out of the cartridge, a user depresses the piston, which breaks the frangible disk and allows the diluent 23 to contact and mix with the medicament 16 to form a solution or suspension

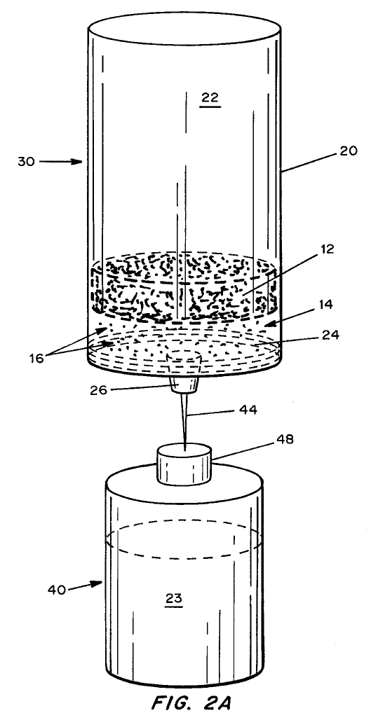

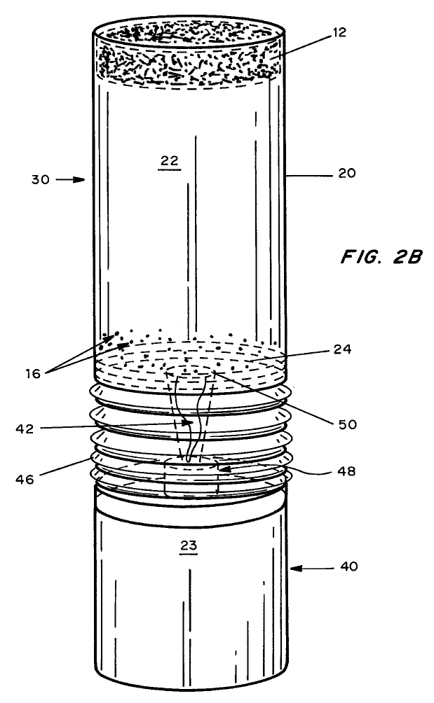

As shown in FIGS. 2A and 2B, in an alternative embodiment, the device 30 can be connected by a tube, such as a nozzle or a needle, to a pre-pressurized container 40 containing the diluent 23. The tube can contain a dispersion or turbulence device (herein referred to as a “dispersion tube”). The dispersion tube has a one-way valve permitting flow of liquid from the pressurized container to the second container.

In the embodiment shown in FIG. 2A, the device 30 consists of a closed, generally tubular system, containing a cartridge 20, which contains a movable piston 12, and a friable chamber 14 containing lyophilized or powdered medicament 16, and a diluent chamber 22. A nozzle 26 and needle 44 are located at the distal end of the cartridge 20. As shown in FIG. 2A, the device 30 connects to the pre-pressurized container 40 containing diluent 23 via the needle 44. In the embodiment shown in FIG. 2B, the device 30 consists of a closed, generally tubular system, containing a cartridge 20, which contains a movable piston 12, a diluent chamber 22 and a lyophilized or powdered medicament 16. A dispersion tube, such as a vortex nozzle 42, is located at the distal end of the cartridge 20. A one-way valve 50 is located at the wide end of the vortex nozzle 42, i.e. the end at which the nozzle attaches to the cartridge 20. The cartridge 20 is connected to the pre-pressurized container 40 using an accordion casing 46. The nozzle 26/needle 44 and the vortex nozzle 42/one-way valve 50 are interchangeable in the device.

The diluent 23 is transferred into the cartridge 20 by pressing the cartridge 20 in a downward direction towards the pre-pressurized container 40; this motion contracts the length of the accordion casing 46 and pushes the vortex nozzle 42 into the top 48 of the pre-pressurized container 40. Alternatively, the diluent 23 is transferred into the cartridge 20 by pushing a needle 44 into the top 48 of the pre-pressurized container 40. The diluent 23 travels out of the pre-pressurized container 40 and into the cartridge 20. The contents of the pressurized container flow turbulently into the cartridge, ensuring thorough mixing. The movable piston is pushed to its full excursion by the incoming fluid. The diluent container and the connecting tube can then separated from the now filled cartridge, containing a solution or suspension comprising the medicament, under sterile conditions. The cartridge 20 can then be inserted in a suitable device for administering the medicament to a patient, such as a pen device.

The devices 10 and 30 are typically made of a material that can be molded into the desired shape and easily sterilized, such as a polycarbonate which can be made by molding and sterilized by exposure to gamma irradiation or ethylene oxide. The piston will typically be formed of a material such as a soft rubber or plastic.

We claim:

1. A tubular device for mixing a single dose of diluent and lyophilized or powdered medicament comprising a piston, friable chamber comprising the lyophilized or powdered medicament, diluent chamber.

2. The device of claim 1 further comprising a nozzle of transfer of diluent to or from the device.

3. The device of claim 1, further comprising a frangible disk between the friable chamber and diluent chamber.

4. The device of claim 3, wherein the piston is located at one end of the device and diluent chamber is located at the other end of the device, and wherein the frangible disk and friable chamber are located between the diluent chamber and the piston.

5. The device of claim 1 wherein the piston is placed in abutment with a friable chamber at a first end of the device, and wherein the first end of device contains a nozzle or needle.

6. The device of claim 5, wherein the nozzle is a vortex nozzle.

7. The device of claim 5, wherein the wide end of the nozzle is attached to a one way valve.

8. The device of claim 5, wherein the device is attached to an accordion casing and pre-pressurized container comprising diluent.

9. A method for storing a medicament in a device comprising
placing a lyophilized or powdered medicament in a tubular device comprising a piston, friable chamber, diluent chamber, and means for separating the medicament from a diluent, wherein the lyophilized or powdered medicament is placed in the friable chamber.

10. The method of claim 9, wherein the device further comprises diluent in the diluent chamber and wherein the means for separating the medicament from a diluent is a frangible disk.

11. The method of claim 9, wherein the means for separating the medicament from a diluent is a pre-pressurized container comprising diluent.

12. A method for mixing a medicament with a diluent in a tubular device, wherein the device comprises a piston, friable chamber, and diluent chamber, wherein the method comprises placing a lyophilized or powdered medicament in the friable chamber of the device, and
adding a diluent to the diluent chamber.

13. The method of claim 12, wherein the device further comprises a frangible disk between the friable chamber and diluent chamber.

14. The method of claim 13, further comprising breaking the frangible disk and allowing the diluent to contact and mix with medicament.

15. The method of claim 12, further comprising attaching a tube to the end of the device, and attaching a pre-pressurized container comprising diluent to the tube.

16. The method of claim 15, wherein the tube is a nozzle or a needle.

17. The method of claim 16, wherein the tube is a nozzle and the device is attached to and accordion casing.

18. The method of claim 17, wherein the step of adding diluent to the diluent chamber comprises pushing the device in a downwards direction towards the pre-pressurized container, and contacting the nozzle with the top of the pre-pressurized container, and allowing the diluent to transfer out of the pre-pressurized container and into the diluent chamber in the device.

19. The method of claim 16, wherein the tube is a needle.
